(19) United States
(12) Patent Application Publication
(10) Pub. No.: US 2024/0001142 A1
(43) Pub. Date: Jan. 4, 2024

(54) INDUCTION BY LOW DOSE RADIATION OF CANCER CELL TARGETS FOR CELL-BASED OR SMALL MOLECULE THERAPY

(71) Applicant: BOARD OF REGENTS, THE UNIVERSITY OF TEXAS SYSTEM, Austin, TX (US)

(72) Inventors: James W. WELSH, Houston, TX (US); Hampartsoum B. BARSOUMIAN, Houston, TX (US); Angelica CORTEZ, Houston, TX (US); Nahum-Puebla OSORIO, Houston, TX (US)

(21) Appl. No.: 18/248,339

(22) PCT Filed: Oct. 7, 2021
(86) PCT No.: PCT/US2021/054051

§ 371 Date: Apr. 7, 2023

Related U.S. Application Data
(60) Provisional application No. 63/088,846, filed on Oct. 7, 2020.

(51) Int. Cl. A61N 5/10; A61K 39/00; A61P 35/00

(52) U.S. Cl. CPC A61N 5/10; A61P 35/00; A61K 39/0011

(57) ABSTRACT

We disclose a method, comprising administering, to a patient suffering from a cancer, low-dose radiation, whereby expression of a target on a surface of a cancer cell increases after the low-dose radiotherapy; and administering, to the patient, a cell-based target-specific cancer therapy, wherein a cell of the cancer therapy interacts with the target. The method may further comprise administering an additional cancer treatment modality. We also disclose a kit comprising a cell of the cell-based target-specific cancer therapy; and instructions to perform the method. Further, we disclose a similar method and corresponding kit, wherein low-dose radiation induces expression of an intracellular target, and a small molecule cancer therapy interacting with the intracellular target is administered.

### FIELD OF THE INVENTION

The present invention relates generally to the field of cancer treatment. More particularly, it concerns the induction by low dose radiation of targets for cancer therapies.

### BACKGROUND OF THE INVENTION

Cell-based cancer therapies, such as chimeric antigen receptor (CAR) T cells, CAR natural killer (NK) cells, and T cell receptor (TCR) engineered T cells, are a relatively new field of cancer therapy with revolutionary potential. In broad strokes, these therapies engineer immune cells, either from the patient or from a healthy donor, to have specificity for targets on the surfaces of cancer cells. These cell-based cancer therapies can greatly enhance the patient's own immune responses against the cancer. Cell-based cancer therapies involving CAR T cells have already been approved by the United States Food & Drug Administration (US FDA) for two blood cancers, acute lymphoblastic leukemia (ALL) and diffuse large B-cell lymphoma (DLBCL).

However, a number of challenges have bedeviled workers attempting to move cell-based cancer therapies from the laboratory to the clinic, especially for non-blood cancers. First, many non-blood cancers present with solid tumors, wherein the surfaces of a majority of the cancer cells are hidden in the tumor mass and the tumor microenvironment is hostile to immune cell activity. These factors reduce the effectiveness of cell-based cancer therapies against many cancers. Second, the target on the surface of the cancer cell should be highly expressed relative to healthy cells, to minimize the collateral damage cell-based cancer therapies may do to the healthy cells. The blood cancers for which CAR T cells have received regulatory approval in the United States are characterized by high relative cell-surface levels of CD19, which is the target for both approved CAR T cell therapies. Finding targets with comparable high relative levels on the surfaces of solid tumor cells has been a slower process.

Accordingly, there is a need for treatment methods using cell-based cancer therapies that have improved specificity against cancer cells, such as non-blood cancers and/or cancers presenting with solid tumors, as opposed to healthy cells.

### SUMMARY OF THE INVENTION

The following presents a simplified summary of the disclosure in order to provide a basic understanding of some aspects of the disclosure. This summary is not an exhaustive overview of the disclosure. It is not intended to identify key or critical elements of the invention or to delineate the scope of the invention. Its sole purpose is to present some concepts in a simplified form as a prelude to the more detailed description that is discussed later.

In one embodiment, the present disclosure relates to a method, comprising administering, to a patient suffering from a cancer, low-dose radiation, whereby expression of a target on a surface of a cancer cell increases after the low-dose radiotherapy; and administering, to the patient, a cell-based target-specific cancer therapy, wherein a cell of the cancer therapy interacts with the target.

In one embodiment, the present disclosure relates to a kit, comprising administering, to a patient suffering from a cancer, low-dose radiation, whereby expression of a target inside a cancer cell increases after the low-dose radiotherapy; and administering, to the patient, a small molecule cancer therapy, wherein the small molecule cancer therapy interacts with the target.

In one embodiment, the present disclosure relates to a kit, comprising: a cell-based target-specific cancer therapy composition, and instructions for the use of the cancer therapy composition in a method comprising administering, to a patient suffering from a cancer, low-dose radiation, whereby expression of a target on a surface of a cancer cell increases after the low-dose radiotherapy; and administering, to the patient, the cell-based target-specific cancer therapy, wherein a cell of the cancer therapy interacts with the target.

In one embodiment, the present disclosure relates to a kit, comprising: a composition comprising a small molecule cancer therapy, and instructions for the use of the composition in a method comprising administering, to a patient suffering from a cancer, low-dose radiation, whereby expression of a target inside a cancer cell increases after the low-dose radiotherapy; and administering, to the patient, the small molecule cancer therapy, wherein the small molecule cancer therapy interacts with the target.

The methods and the kits of the present disclosure may grant cell-based cancer therapies improved specificity against cancer cells.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 presents a flowchart of a first method in accordance with embodiments herein.

FIG. 2 presents a flowchart of a second method in accordance with embodiments herein.

FIG. 3 shows a Mann-Whitney test of ranks in expression levels before (pre) and after (post) administration of low-dose radiation in Example 1.

FIG. 4 shows a heat map of gene expression before and after administration of low-dose radiation for 66 genes in Example 1.

FIG. 5 presents the two low-dose radiation regimens used in Example 3.

FIG. 6A presents a heat map of expression differences in the GSU gastric carcinoma cell line for seven genes at day 3 of the negative control, single low-dose radiation (LD-XRT) and pulsed low-dose radiation (Pulsed-LD-XRT) regimens described in Example 3.

FIG. 6B presents a bar graph of relative expression differences in the GSU gastric carcinoma cell line for seven genes at day 3 of the negative control, LD-XRT, and PulsedLD-XRT regimens described in Example 3.

FIG. 7A presents a heat map of expression differences in the GSU gastric carcinoma cell line for seven genes at day 6 of the negative control, LD-XRT, and Pulsed-LD-XRT regimens described in Example 3.

FIG. 7B presents a bar graph of relative expression differences in the GSU gastric carcinoma cell line for seven genes at day 6 of the negative control, LD-XRT, and PulsedLD-XRT regimens described in Example 3.

FIG. 8A presents a heat map of expression differences in the A549 lung adenocarcinoma cell line for seven genes at day 3 of the negative control, LD-XRT, and Pulsed-LD-XRT regimens described in Example 3.

FIG. 8B presents a bar graph of relative expression differences in the A549 lung adenocarcinoma cell line for seven genes at day 3 of the negative control, LD-XRT, and PulsedLD-XRT regimens described in Example 3.

FIG. 9A presents a heat map of expression differences in the A549 lung adenocarcinoma cell line for seven genes at day 6 of the negative control, LD-XRT, and Pulsed-LD-XRT regimens described in Example 3.

FIG. 9B presents a bar graph of relative expression differences in the A549 lung adenocarcinoma cell line for seven genes at day 6 of the negative control, LD-XRT, and PulsedLD-XRT regimens described in Example 3.

FIG. 10A presents a heat map of expression differences in the Flo1 esophageal carcinoma cell line for seven genes at day 3 of the negative control, LD-XRT, and Pulsed-LD-XRT regimens described in Example 3.

FIG. 10B presents a bar graph of relative expression differences in the Flo1 esophageal carcinoma cell line for seven genes at day 3 of the negative control, LD-XRT, and PulsedLD-XRT regimens described in Example 3.

FIG. 11A presents a heat map of expression differences in the Flo1 esophageal carcinoma cell line for seven genes at day 6 of the negative control, LD-XRT, and Pulsed-LD-XRT regimens described in Example 3.

FIG. 11B presents a bar graph of relative expression differences in the Flo1 esophageal carcinoma cell line for seven genes at day 6 of the negative control, LD-XRT, and PulsedLD-XRT regimens described in Example 3.

While the subject matter disclosed herein is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims. Moreover, the stylized depictions illustrated in the drawings are not drawn to any absolute scale.

### DESCRIPTION OF ILLUSTRATIVE EMBODIMENTS

Various illustrative embodiments of the disclosure are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related, regulatory, and business-related constraints, which will vary from one implementation to another. Moreover, it will be appreciated that such a development effort might be complex and time-consuming but would nevertheless be a routine undertaking for those of ordinary skill in the art having the benefit of this disclosure.

The present subject matter will now be described with reference to the attached figures. Various structures, systems, and devices are schematically depicted in the drawings for purposes of explanation only and so as to not obscure the present disclosure with details that are well known to those skilled in the art. Nevertheless, the attached drawings are included to describe and explain illustrative examples of the present disclosure. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, any given numerical value includes the inherent variation of error for the device, or the method being employed to determine the value, or the variation that exists between study subjects or healthcare practitioners.

Embodiments herein provide for cell-based cancer therapies that may deliver improved specificity against cancer cells. In some embodiments, the cell-based cancer therapies may provide specificity against non-blood cancers and/or cancers presenting with solid tumors, as opposed to healthy cells.

Embodiments herein provide for facilitating an increase in expression of a cell surface target in a cancer patient's body. A cell-based, target-specific cancer-related therapy may be then administered. This process may, if desired, be followed with an alternative cancer treatment.

Embodiments herein provide for facilitating an increase in expression of an intracellular target in a cancer patient's body. A cancer-related therapy specific for the intracellular target, such as a small molecule cancer therapy, may be then administered. This process may, if desired, be followed with an alternative cancer treatment.

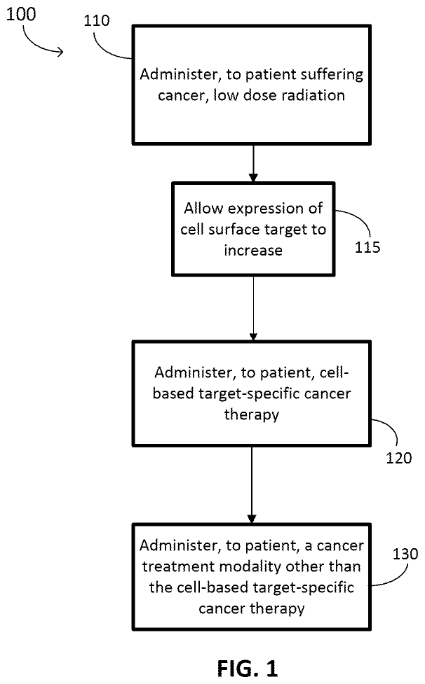

FIG. 1 presents a flowchart of a method 100 in accordance with embodiments of the present disclosure. The method 100 comprises administering (at 110), to a patient suffering from a cancer, low-dose radiation. This process may be followed by a process for allowing an increase of expression of a target on a surface of a cancer cell after the low-dose radiotherapy (at 115). A cell-based, target-specific cancer therapy may then be administered (at 120), wherein a cell of the cancer therapy interacts with the target.

The patient may be any mammal suffering from the cancer. In one embodiment, the patient is a human being.

In embodiments, the present method may be performed in a veterinary context. That is, the patient may be any non-human mammal suffering from a cancer. The non-human mammal may be a research animal, a pet, livestock, a working animal, a racing animal (e.g., a horse, a dog, a camel, etc.), an animal at stud (e.g., a bull, a retired racing stallion, etc.), or any other non-human mammal for which it is desired to treat its cancer.

For convenience, the description will typically refer to human patients. However, the person of ordinary skill in the art having the benefit of the present disclosure will readily be able to adapt the teachings of the present disclosure to a veterinary context.

By “suffering from a cancer” is meant that the cancer is detectable in the patient's body using any diagnostic technique presently known or to be discovered. “Suffering” does not require the patient to be in pain from or have any naturally-perceptible symptoms of the cancer. Generally, as is known, the earlier a cancer can be treated, including before the patient notices pain or any other symptoms, the greater the chances of remission.

The present method may be used to treat any type of cancer. Desirably, the cancer is one that is known or reasonably expected, by the person of ordinary skill in the art having the benefit of the present disclosure, to be treatable by a cell-based target-specific cancer therapy.

In one embodiment, the cancer is characterized by a solid tumor. Not every presentation of the type of the cancer must feature a solid tumor for the cancer to be “characterized by a solid tumor.” If the person of ordinary skill in the art knows as a routine matter that the type of cancer often presents with a solid tumor, then the cancer is “characterized by a solid tumor.”

For the avoidance of doubt, the method 100 may be performed at any stage of the cancer, including after metastasis, when it is desirable to kill cancer cells outside of a solid tumor.

In the method 100, low-dose radiation is administered (at 110) to the patient. Although not strictly radiation therapy, the low-dose radiation may be administered (at 110) following general principles established for radiation therapy. Generally, radiation comprising particles (such as protons, carbon, etc.) or photons that have sufficient energy or can produce sufficient energy via nuclear interactions is aimed at cancer cells to produce ionization (i.e., loss of electrons) in the cancer cells. In response, and in addition to other chemical and biochemical processes, the cancer cells may increase or decrease the expression of one or more genes in an effort to repair radiation damage. An exemplary ionizing radiation is X-ray radiation. Apparatus and techniques for delivering X-rays to a target tissue or cell are well known in the art.

“Low-dose” generally means a radiation dose that is below an amount that the person of ordinary skill in the art would expect to be directly efficacious against the cancer. Whether a dose is below the amount expected to be directly efficacious may depend on the type of cancer, the size of the tumor, the location in the body of the tumor, etc.

In one embodiment, the administering (at 110) comprises delivering from one to five doses of radiation, wherein each dose of radiation is from about 10 cGy to about 150 cGy. In a particular embodiment, the administering the low-dose radiation comprises delivering from one to five doses of radiation, wherein each dose of radiation is from about 10 cGy to about 150 cGy. These doses are below typical total doses for radiation therapy, which can be as high as 6000 cGy-7000 cGy.

As stated, administering (at 110) is expected to increase the expression of one or more genes. (Herein, the terms “expression of a gene” and “expression of a protein” are used interchangeably to refer to the production of the protein encoded by the gene). One or more of these genes may encode a protein on a surface of the cancer cell. Such a protein may be a target for a cell-based target-specific cancer therapy to be discussed later.

Particular targets may vary depending on the particular cancer and/or various parameters of the administering (at 110) of the low-dose radiation. In one embodiment, the target is selected from the group consisting of SCARB2, SERINC1, IL6ST, IL6R, XPC, ITGAV, MR1, and DCT.

In one embodiment, the target is SCARB2 (scavenger receptor class B member 2, also known as CD36L2). Though not to be bound by theory, SCARB2 is currently best understood as a lysosomal receptor for glucosylceramidase (GBA) targeting.

In one embodiment, the target is SERINC1 (serine incorporator 1). Though not to be bound by theory, SERINC1 is currently best understood as enhancing the incorporation of serine into phosphatidylserine and sphingolipids in the cell membrane.

In one embodiment, the target is IL6ST (interleukin 6 signal transducer). Though not to be bound by theory, binding of interleukin 6 (IL6) to IL6 receptor (IL6R), as currently best understood, induces homodimerization of IL6ST and formation of a high-affinity receptor complex, which activates Janus kinases.

In one embodiment, the target is IL6R (IL6 receptor). Though not to be bound by theory, as currently best understood, IL6R requires association with IL6ST for signal activation.

In one embodiment, the target is XPC (XPC complex subunit). Though not to be bound by theory, as currently best understood, XPC is a DNA damage recognition and repair factor.

In one embodiment, the target is ITGAV (integrin subunit alpha V, also known as CD51). Though not to be bound by theory, as currently best understood, ITGAV mediates the release of TGF-β.

In one embodiment, the target is MR1. Though not to be bound by theory, as currently best understood, MR1 is a non-polymorphic MHC-I-like protein that is expressed at low to undetectable levels on the surface of many cell types.

In one embodiment, the target is DCT (dopachrome tautomerase). Though not to be bound by theory, as currently best understood, DCT is involved in regulating eumelanin and phaeomelanin levels.

In one embodiment, the target is selected from the group consisting of BCAN, CD130, CD51, DCT, interleukin 6 receptor (IL-6R), major histocompatabililty complex class I-related (MR1), and xeroderma pigmentosum, complementation group C (XPC).

In one embodiment, the target is BCAN.

In one embodiment, the target is CD130.

The processes required for a cancer cell to respond to administering (at 110) by increasing the expression of a cell surface target protein may require seconds, minutes, hours, or days, depending on the particular cell surface target protein, the particular type of cancer cell, the radiation intensity, dose number, and dose frequency of the administering (at 110), etc., as will be known to the person of ordinary skill in the art having the benefit of the present disclosure. The method 100 may accordingly comprise allowing (at 115) the expression of the cell surface target to increase. However, in some situations, the expression of a given target may increase enough in a rapid enough timeframe that no explicit “allowing” (at 115) need be performed.

The “administering” (at 110) and the “allowing” (at 115), if performed, are expected to result in the cell surfaces of at least a portion of the cancer cells becoming enriched in the target. Accordingly, cell-based cancer therapies specific for the target may have increased efficacy against at least the portion of the cancer cells. This could be considered a “let a hundred flowers bloom” strategy of cancer therapy, analogous to the policy of Mao Zedong of encouraging free expression in China in 1956-1957, which only exposed dissidents to subsequent Maoist suppression in 1957-1959.

It should be appreciated that, absent the non-natural induction of expression by low-dose radiation, the target would generally not be a suitable target for a cell-based cancer therapy, because the pre-radiation levels of the target on the surface of the cancer cells are unlikely to be significantly higher than the levels on noncancerous cells. Use of cell-based cancer therapies against non-induced targets would be expected to lead to systemic side effects that, at minimum, are undesirable, and maybe even harmful. These concerns could increase the probability of rejection by the United States Food and Drug Administration (FDA) and/or other regulatory agencies on patient safety grounds of one or more such therapies. The present method is expected to enhance patient safety and/or efficacy by unnaturally enriching cancer cell surfaces with the target.

The method 100 further comprises administering (at 120), to the patient, a cell-based target-specific cancer therapy, wherein a cell of the cancer therapy interacts with the target.

Cell-based cancer therapies are a relatively new paradigm in cancer treatment, though one that the person of ordinary skill in the art having the benefit of the present disclosure will be knowledgeable of. Generally, immune cells are derived from the patient or a healthy subject, engineered to be specific for a protein expressed on the surface of a cancer cell, and then administered to the patient. The immune cells then interact with the protein, whereupon they may attack and/or kill the cancer directly or indirectly by stimulating the patient's immune system to attack and destroy the cancer.

In one embodiment, the cell of the cell-based target-specific cancer therapy is a chimeric antigen receptor (CAR) T cell. A CAR T cell is a T cell genetically engineered to express a CAR, which is a chimera of an extracellular antigen-recognition domain engineered or derived to be specific for a target protein, and an intracellular T cell-activating domain that activates the CAR T cell's T cell functionality after the antigen-recognition domain binds to the target protein.

In one embodiment, the cell of the cell-based target-specific cancer therapy is a CAR natural killer (NK) cell. The processes of forming and using a CAR NK cell are similar to those of forming a CAR T cell, with the modifications necessary to apply these processes to NK cells being a routine matter for the person of ordinary skill in the art having the benefit of the present disclosure.

In one embodiment, the cell of the cell-based target-specific cancer therapy is a T cell receptor (TCR) engineered T cell. T cells naturally present TCR protein complexes on their surfaces which recognize antigen fragments bound to major histocompatibility complexes (MHCs), but as is known, TCRs have low specificity for any given antigen. Engineering of the TCR can increase the specificity of that TCR for a given antigen, e.g., a target protein or fragment thereon, and a T cell presenting the engineered TCR may have increased efficacy against that antigen without a concomitant increase in activity against other antigens.

The cell-based target-specific cancer therapy may be prepared by any appropriate technique known to those skilled in the art having benefit of the present disclosure. In an exemplary preparation scheme, antibodies for cellular targets increased by low-dose radiotherapy may be generated by known techniques. The sequences of the single chain fragment variables (scFvs) of the antibodies may be obtained, followed by the generation of plasmid or lentiviral vectors containing sequences encoding for the expression of a chimeric antigen receptor specific for a given target. Routine quality control measures, such as in vitro testing for the expression of the antigen receptors in recipient T cells, measuring the expression of the vector by flow cytometry, assessing T-cell-target killing assays, and preclinical testing using model organisms and/or cell cultures, such as syngeneic mouse models or the PDX pancreatic cancer model developed at our institution.

In one embodiment of administering (at 120) in the method 100, the cell of the cell-based target-specific cancer therapy is selected from the group consisting of chimeric antigen receptor (CAR) T cells, CAR natural killer (NK) cells, T cell receptor (TCR) engineered T cells, and two or more thereof.

The cell-based target-specific cancer therapy may be administered (at 120) to the patient by any appropriate route known to those skilled in the art having benefit of the present disclosure. In one embodiment, administering (at 120) the cell-based target-specific cancer therapy comprises injection of the therapeutic cells in proximity to malignant cells of the cancer. For example, administering (at 120) may comprise injecting the therapeutic cells into the tumor. Other techniques for administering (at 120) the cell-based target-specific cancer therapy will be known to the person of ordinary skill in the art having the benefit of the present disclosure.

In the method 100, administering (at 120) the cell-based target-specific cancer therapy may be performed in a single dose or a plurality of doses. The number of doses, frequency of doses, and cell count of doses are matters of routine experimentation for the person of ordinary skill in the art having the benefit of the present disclosure and need not be described in detail.

The method 100 may comprise additional events. In one embodiment, the method 100 may further comprise administering (at 130), to the patient, an additional cancer treatment modality other than the cell-based target-specific cancer therapy. Administering (at 130) the additional cancer treatment modality other than the cell-based target-specific cancer therapy may be targeted against the same cancer as the cell-based target-specific cancer therapy, against metastases thereof, against a primary tumor or metastases of a cancer other than cancer targeted by the cell-based target-specific cancer therapy, or two or more thereof.

A wide variety of cancer treatment modalities other than cell-based target-specific cancer therapy are known to the person of ordinary skill in the art and need not be described in detail here. By way of example, in one embodiment, the additional cancer treatment modality apart from the cell-based target-specific cancer therapy is selected from the group consisting of surgical resection, chemotherapy, immunotherapy, checkpoint inhibitor therapy, oncolytic virus therapy, thermal therapy (e.g., RFA, microwave ablation, and/or cryotherapy), and two or more thereof.

In a particular embodiment, the additional cancer treatment modality may be checkpoint inhibitor therapy, such as anti-PD1 therapy, anti-PDL1 therapy, anti-TIGIT therapy, anti-GITR therapy, and anti-CTLA-4 therapy, among others.

Regardless of the particular cancer treatment modality other than cell-based target-specific cancer therapy, if one or more is/are administered (at 130), the administering (at 130) may be performed before, after, or simultaneously with the administering (at 120) the cell-based target-specific cancer therapy. Particular relative and absolute timing of administering (at 120) the cell-based target-specific cancer therapy and administering (at 130) the other cancer treatment modality will be a routine matter for the person of ordinary skill in the art having the benefit of the present disclosure.

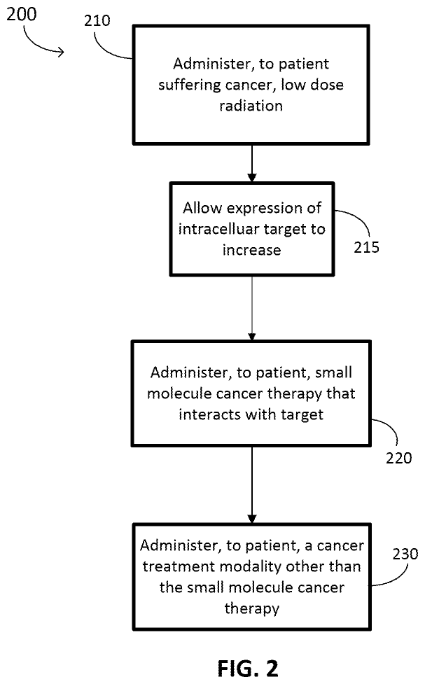

Turning to FIG. 2, a flowchart is presented of a second method 200 in accordance with embodiments herein. Many of the actions performed in the second method 200 are similar to those performed in the first method 200. Accordingly, the following description of the second method 200 will focus on differences between the two methods.

The method 200 comprises administering (at 210), to a patient suffering from a cancer, low-dose radiation, whereby expression of a target inside a cancer cell increases after the low-dose radiotherapy. Administering (at 210) is substantially the same as administering (at 110 of first method 100).

As a result of administering (at 210), the expression of one or more intracellular proteins by the cancer cells may increase. The expression may be explicitly allowed (at 215) to increase, but need not be.

In the second method 200, wherein the target is selected from the group consisting of CDKN1A (p21), DNAJB4, FNIP2, GPRASP2, GSKIP, GUCD1, HIST1H2AC, HIST1H2BC, KLF11, LIG4, MDM2, and NBR1.

In one embodiment, the target is CDKN1A (p21).

In one embodiment, the target is DNAJB4.

In one embodiment, the target is FNIP2.

In one embodiment, the target is GPRASP2.

In one embodiment, the target is GSKIP.

In one embodiment, the target is GUCD1.

In one embodiment, the target is HIST1H2AC.

In one embodiment, the target is HIST1H2BC.

In one embodiment, the target is KLF11.

In one embodiment, the target is LIG4.

In one embodiment, the target is MDM2.

In one embodiment, the target is NBR1.

A difference between the first method 100 and the second method 200 can be seen in administering (at 220), to the patient, a small molecule cancer therapy, wherein the small molecule cancer therapy interacts with the target. Because the target in the second method 200 is not presented on the surface of cancer cells, the cell-based target-specific cancer therapies of the first method 100 would not be effective against it. Instead, small molecule cancer therapies that are specific for the intracellular target and capable of entering cancer cells are administered (at 220) in the second method 200.

Small molecules that may have efficacy in treating cancer by modulating intracellular targets, such as CDKN1A (p21)_{1,2}, DNAJB4^{3,4}, FNIP2⁵, GPRASP2⁶, GSKIP, GUCD1, HIST1H2AC, HIST1H2BC, KLF11, LIG4⁷, MDM2^{8,9}, and NBR1¹⁰, are known to the person of ordinary skill in the art having the benefit of the present disclosure. 1: Kreis, N. N., Louwen, F. & Yuan, J. The Multifaceted p21 (Cip1/Waf1/CDKN1A) in Cell Differentiation, Migration and Cancer Therapy. Cancers (Basel) 11, doi:10.3390/cancers11091220 (2019). 2: Lazzarini, R. et al. Enhanced antitumor therapy by inhibition of p21waf1 in human malignant mesothelioma. Clin Cancer Res 14, 5099-5107, doi:10.1158/1078-0432.CCR-08-0255 (2008). 3: Chatterjee, S. & Burns, T. F. Targeting Heat Shock Proteins in Cancer: A Promising Therapeutic Approach. Int J Mol Sci 18, doi:10.3390/ijms18091978 (2017). 4: Acun, T. et al. Hill (DNAJB4) Gene Is a Novel Biomarker Candidate in Breast Cancer. OMICS 21, 257-265, doi:10.1089/omi.2017.0016 (2017). 5: Hasumi, H. et al. Folliculin-interacting proteins Fnip1 and Fnip2 play critical roles in kidney tumor suppression in cooperation with Flcn. Proc Natl Acad Sci USA 112, E1624-1631, doi:10.1073/pnas.1419502112 (2015). 6: Jung, B. et al. Novel small molecules targeting ciliary transport of Smoothened and oncogenic Hedgehog pathway activation. Sci Rep 6, 22540, doi:10.1038/srep22540 (2016). 7: Huang, R. X. & Zhou, P. K. DNA damage response signaling pathways and targets for radiotherapy sensitization in cancer. Signal Transduct Target Ther 5, 60, doi:10.1038/s41392-020-0150-x (2020). 8: Shaikh, M. F. et al. Emerging Role of MDM2 as Target for Anti-Cancer Therapy: A Review. Ann Clin Lab Sci 46, 627-634 (2016). 9: Meek, D. W. & Hupp, T. R. The regulation of MDM2 by multisite phosphorylation—opportunities for molecular-based intervention to target tumours? Semin Cancer Biol 20, 19-28, doi:10.1016/j.semcancer.2009.10.005 (2010). 10: Marsh, T. et al. Autophagic Degradation of NBR1 Restricts Metastatic Outgrowth during Mammary Tumor Progression. Dev Cell 52, 591-604 e596, doi:10.1016/j.devcel.2020.01.025 (2020). The second method 200 may also comprise administering (at 230), to the patient, an additional cancer treatment modality other than the small molecule cancer therapy. The additional cancer treatment modality other than the small molecule cancer therapy may be selected from the group consisting of surgical resection, immunotherapy, checkpoint inhibitor therapy, oncolytic virus therapy, thermal therapy, and two or more thereof.

In a particular embodiment, the first method 100 and the second method 200 may be performed simultaneously. In other words, a single regime of low dose radiation may satisfy both the administering (at 110) element of the first method 100 and the administering (at 210) element of the second method 200. The single regime of low dose radiation may increase expression of both a cell surface target protein, which may be the target of the cell-based target-specific cancer therapy administered (at 120) in the first method 100, and an intracellular target protein, which may be the target of the small molecule cancer therapy administered (at 220) in the second method 200. Considered differently, the administering (at 220) may satisfy the administering (at 130) an additional cancer treatment modality other than the cell-based target-specific cancer therapy element of the first method 100, and/or the administering (at 120) may satisfy the administering (at 230) an additional cancer treatment modality other than the small molecule cancer therapy element of the second method 200.

In one embodiment, the present disclosure relates to a first kit, comprising a cell-based target-specific cancer therapy composition, and instructions for the use of the cancer therapy composition in a method comprising administering, to a patient suffering from a cancer, low-dose radiation, whereby expression of a target on a surface of a cancer cell increases after the low-dose radiotherapy; and administering, to the patient, the cell-based target-specific cancer therapy, wherein a cell of the cancer therapy interacts with the target.

A “kit,” as used herein, refers to a package containing the composition, and instructions of any form that are provided in connection with the composition in a manner such that a clinical professional will clearly recognize that the instructions are to be associated with the composition.

“Instructions” typically involve written text and/or graphics on or associated with packaging of compositions of the disclosure. Instructions also can include any oral or electronic instructions provided in any manner. Written text and/or graphics may include a website URL or a QR code encoding a website URL, where other instructions or supplemental information may be provided in electronic form.

The first kit may contain one or more containers, which can contain the composition or a component thereof. The first kit also may contain instructions for storing, preparing, mixing, diluting, or administering the composition. The first kit also can include other containers with one or more solvents, surfactants, preservatives, and/or diluents (e.g., normal saline (0.9% NaCl), or 5% dextrose) as well as containers for mixing, diluting, or administering the composition to the patient in need of such treatment.

The composition may be provided in any suitable form, for example, as a liquid solution, a frozen solution, or as a lyophilized or otherwise reconstitutable material. When the composition provided is a dry material, the material may be reconstituted by the addition of solvent, which may also be provided by the first kit. In embodiments where liquid or frozen forms of the composition are used, the form may be concentrated, thereby requiring dilution, or ready to use.

The first kit, in one embodiment, may comprise a carrier being compartmentalized to receive in close confinement one or more containers such as vials, tubes, and the like.

The composition comprises one or more cells of the cell-based target-specific cancer therapy, as described above. The composition may also comprise additional components that will be apparent to the person of ordinary skill in the art having the benefit of the present disclosure.

The method is described above. In one embodiment, the instructions comprise instructions to administer the composition by injection of the composition in proximity to the cancer. Alternatively, or in addition, in one embodiment, the instructions comprise instructions to administer the radiation by administering X-rays. Again, alternatively or in addition, in one embodiment, the instructions further comprise instructions to administer, to the patient, an additional cancer treatment modality other than the cell-based target-specific cancer therapy. In further embodiments, the first kit may further comprise active and/or inactive agents of the additional cancer treatment modality.

Similarly, in one embodiment, the present disclosure relates to a second kit, comprising a composition comprising a small molecule cancer therapy, and instructions for the use of the composition in a method comprising administering, to a patient suffering from a cancer, low-dose radiation, whereby expression of a target inside a cancer cell increases after the low-dose radiotherapy; and administering, to the patient, the small molecule cancer therapy, wherein the small molecule cancer therapy interacts with the target. The specific details of the second kit can be implemented as a routine matter by the person of ordinary skill in the art having the benefit of the present disclosure, including the description of the first kit, above.

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

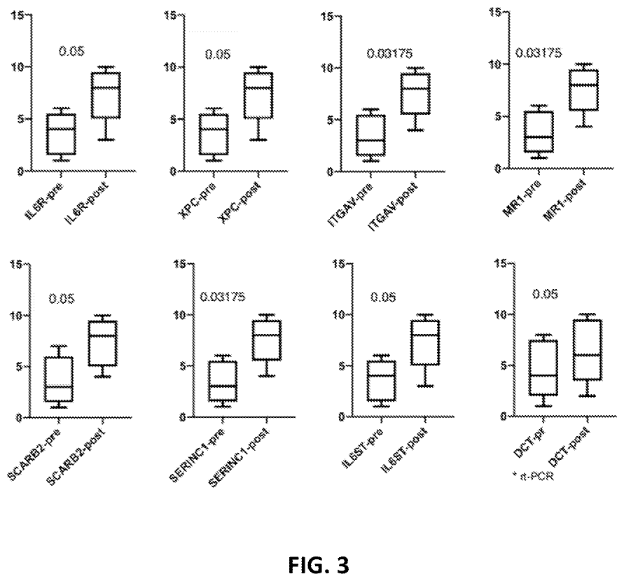

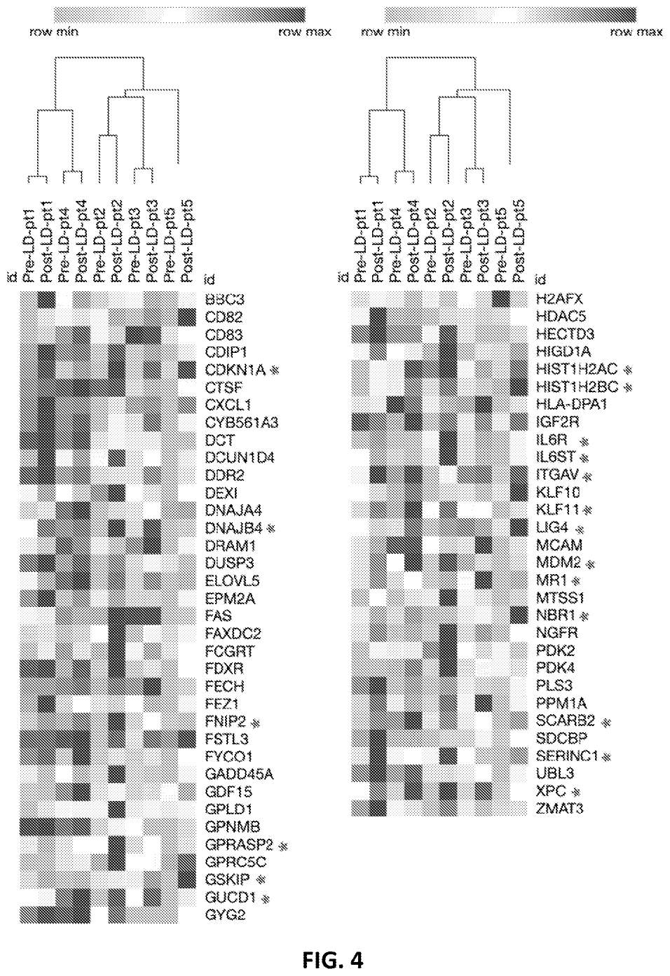

We analyzed multiple biopsies from patients with varied solid tumors before low-dose and post-low-dose radiotherapy. We generated RNA libraries and sent the samples to a third party for RNA sequencing. The raw RNA sequence data were analyzed by a bioinformatician. Out of a potential 13,000 targets, we selected 817 markers with the most significant p-values in their expression levels (p<0.05). We selected 66 targets that showed 95% confidence in increased expression subsequent to low-dose radiotherapy in all patients (FIG. 4). To understand the difference in the expression levels due to low-dose irradiation, we performed rank test analysis between pre vs post radiotherapy on each of the 66 markers and selected 19 targets that showed significant differences (p<0.05). Because optimal cellular targets for T/NK cell engineering are expressed on the cell membrane, we selected eight markers whose expression is predominantly on the cell membrane (FIG. 3). In summary, the membrane targets modulated by low-dose radiotherapy include SCARB2, SERINC1, IL6ST, IL6R, XPC, ITGAV, MR1, and DCT; in addition, we observed a significant induction of other targets that could potentially be used for small molecule inhibition, those include: CDKN1A (p21), DNAJB4, FNIP2, GPRASP2, GSKIP, GUCD1, HIST1H2AC, HIST1H2BC, KLF11, LIG4, MDM2, and NBR1.

### Example 2

To build on the findings of Example 1 in a controlled lung cancer setting, we established human H460 lung adenocarcinoma tumors in nude mice lacking adaptive immunity. We implanted one million tumor cells in the right hind leg of nude mice. When the tumors reached a diameter of 7-8 mm, we delivered local low-dose radiation, as follows:

Treatment

Control, no radiation
Low-dose radiation, 1 Gy × 2
Low-dose radiation, 1.4 Gy × 3

We generated RNA libraries from harvested tumors three days after the last fraction of low-dose radiation. We designed PCR primers for 27 preselected human genes coding for membrane proteins and performed RT-PCR. The 1 Gy×2 treatment group exhibited an increased level of Brevican (BCAN, chondroitin sulfate proteoglycan 7) and Tetraspanin 10 (TSPAN10) proteins over the control group (P<0.07).

### Example 3

We conducted an in vitro study of expression of low-dose X-radiation (LD-XRT) for seven marker genes in three cancer cell lines. The marker genes used were BCAN, CD130, CD51, dopachrome tautomerase (DCT), interleukin 6 receptor (IL-6R), major histocompatabililty complex class I-related (MR1), and xeroderma pigmentosum, complementation group C (XPC). The cell lines used were A549 lung adenocarcinoma; GSU gastric carcinoma; and Flo1 esophageal carcinoma. Cell lines were maintained and gene expression determined using standard techniques.

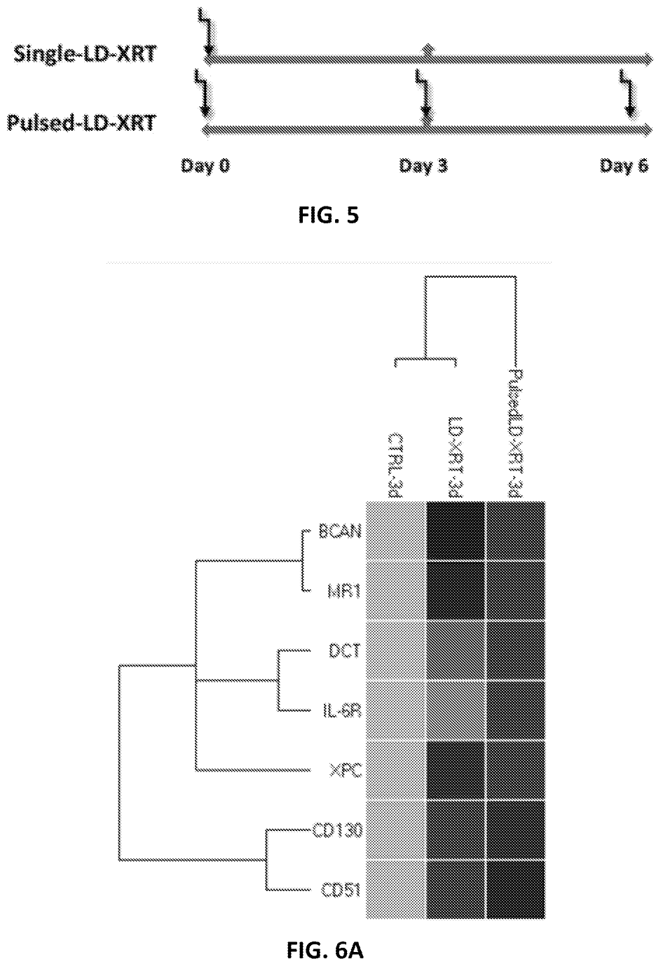

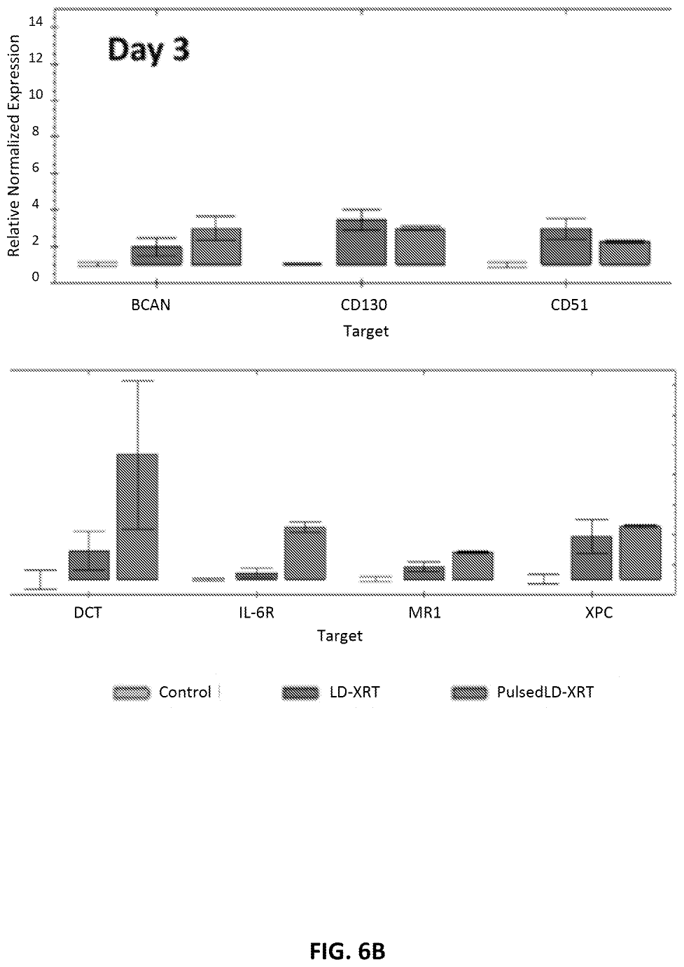

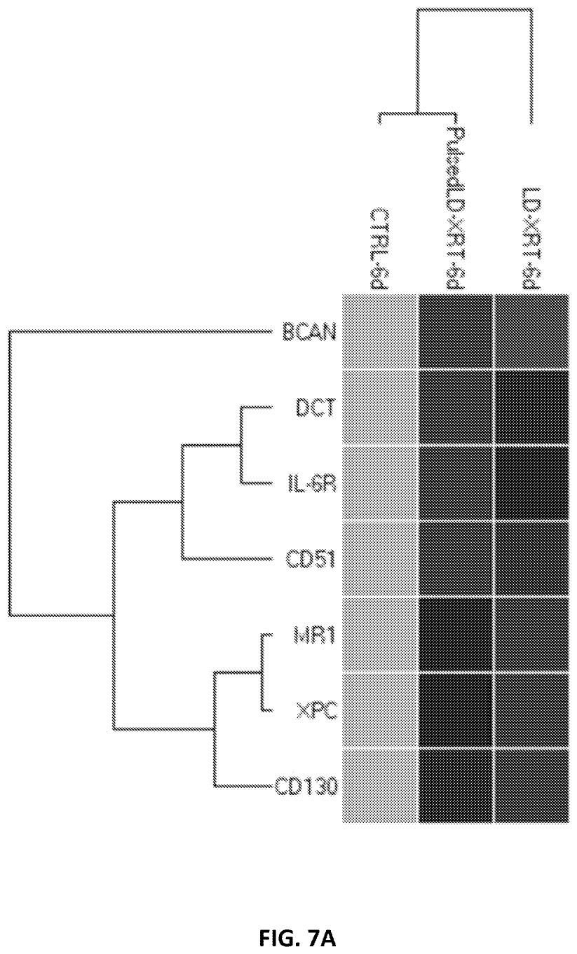

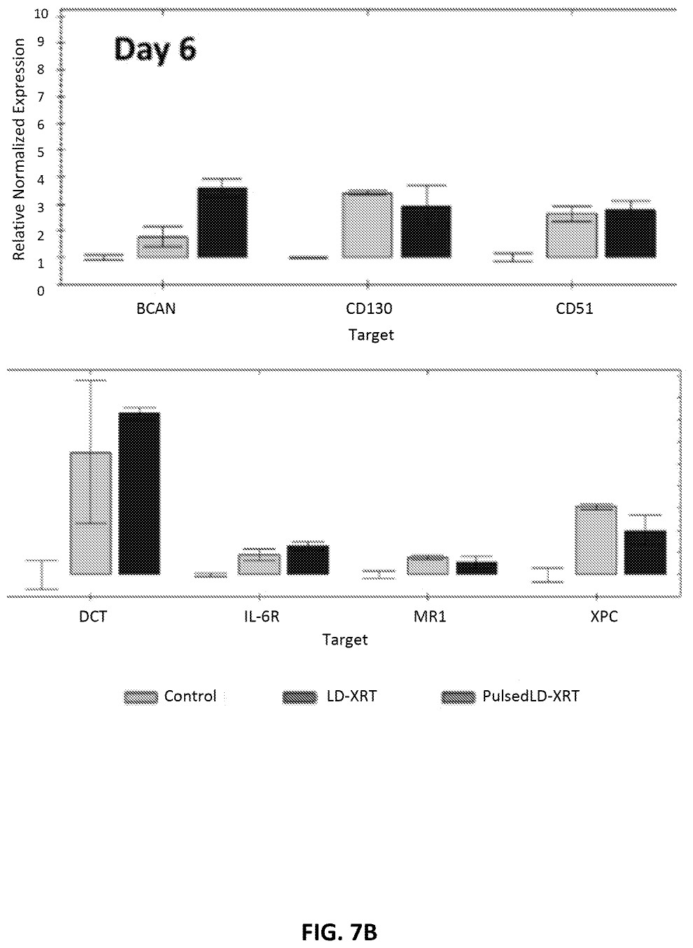

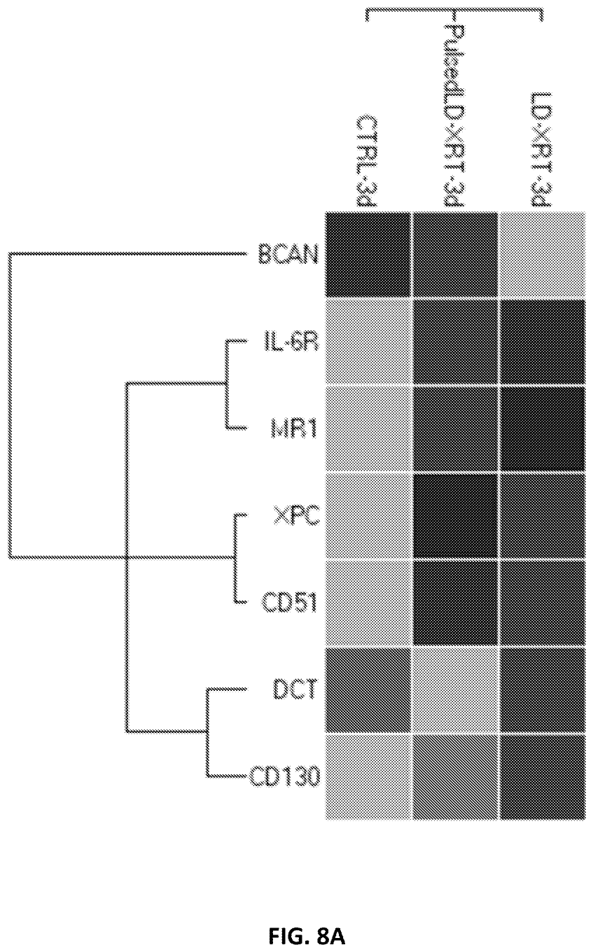

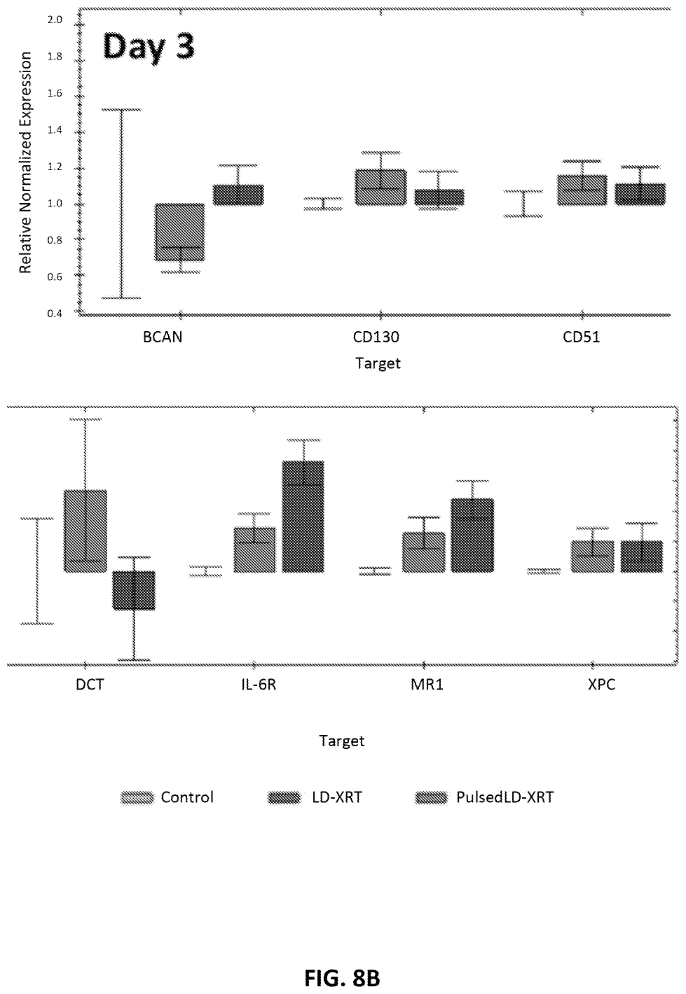

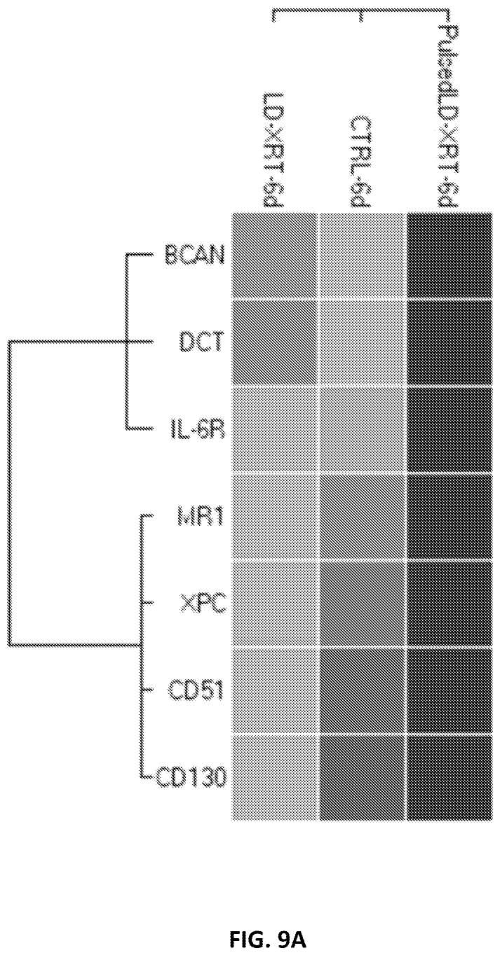

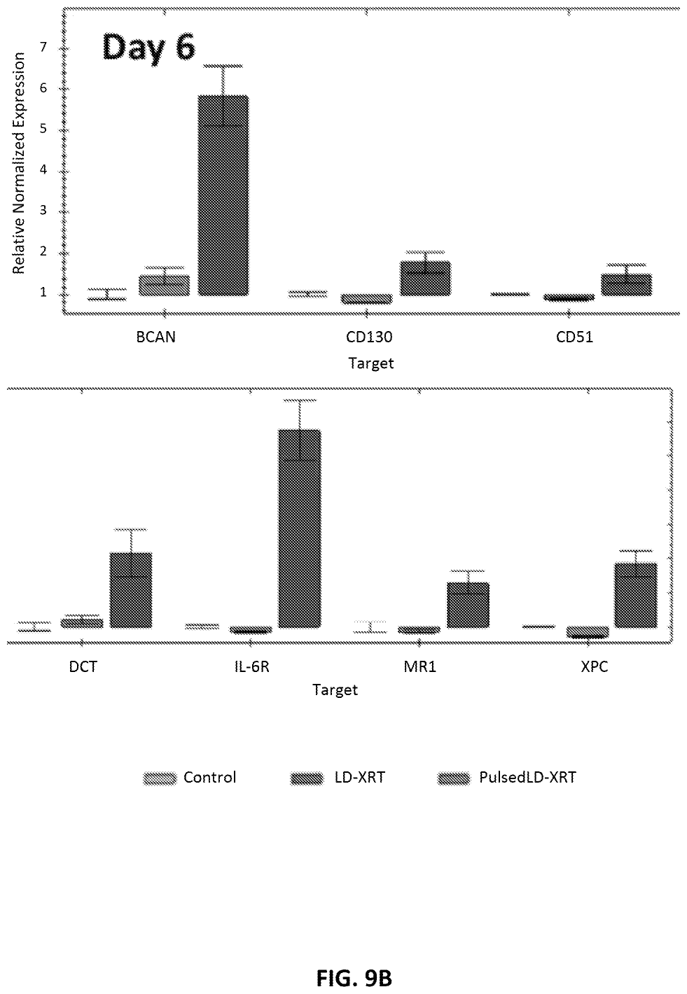

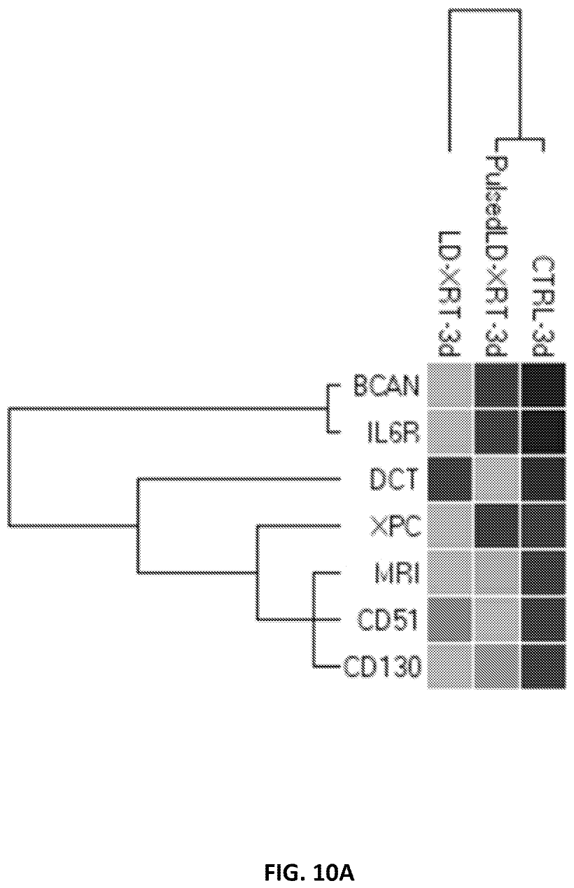

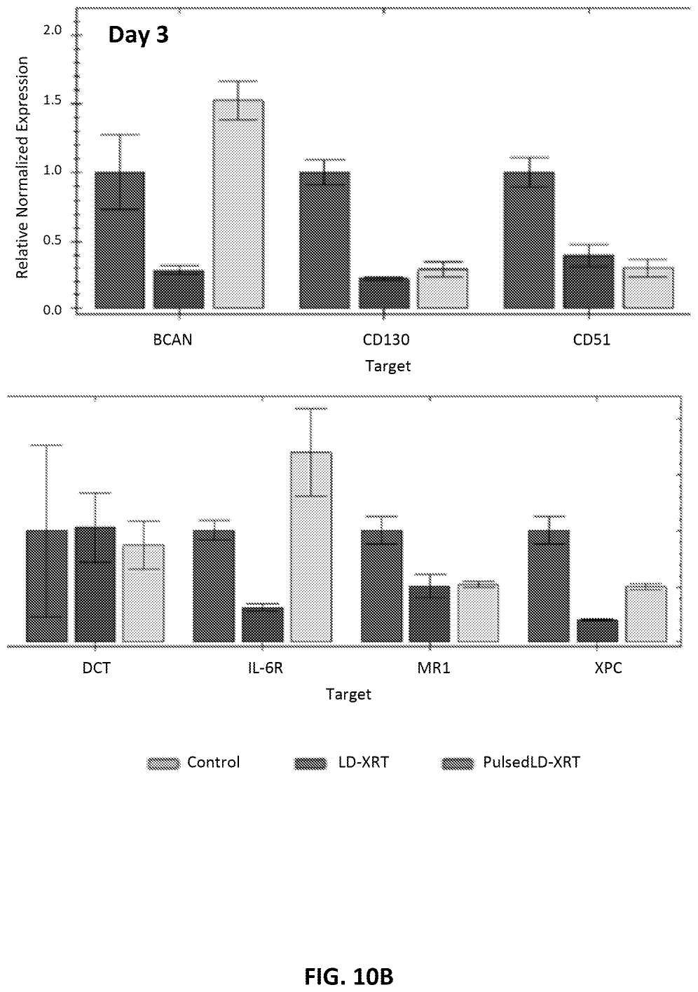

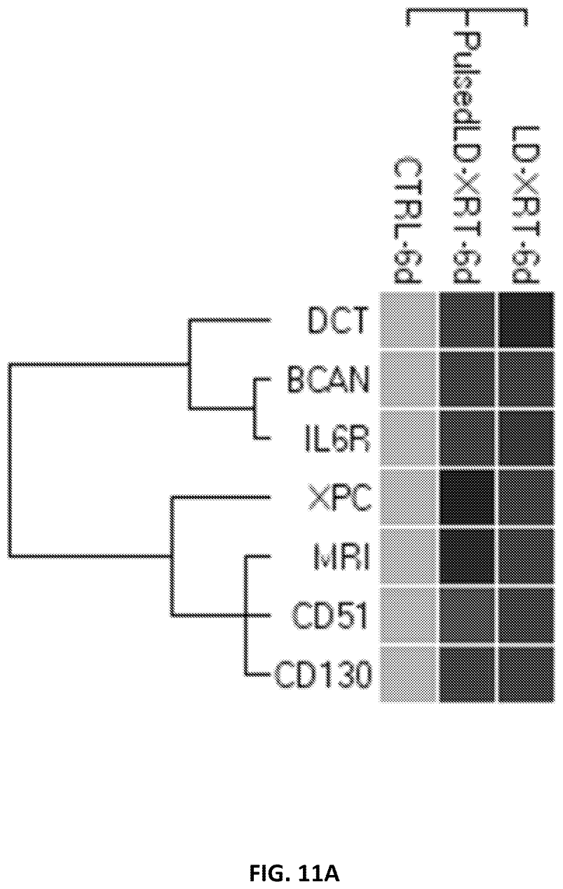

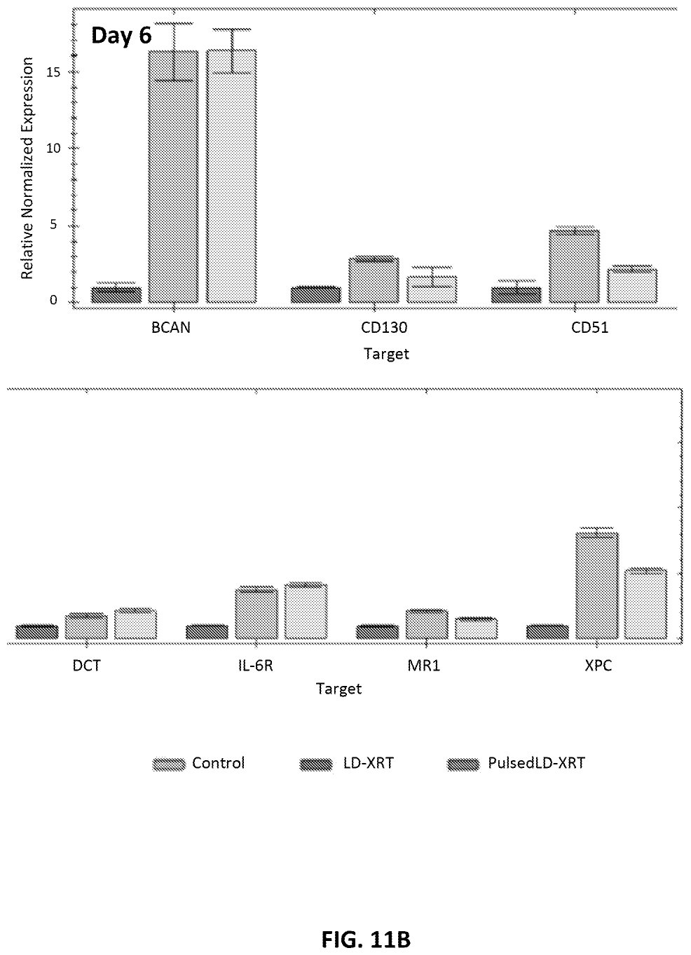

Each of the cell lines was divided into three groups, a negative control, a group subjected to a single 1 Gy dose of ionizing X-rays (LD-XRT), and a group subjected to pulses of low-dose radiation at a regimen of one dose every three days. In FIG. 5-FIG. 11B, these three groups are identified as Control, LD-XRT, and PulsedLD-XRT, respectively. Cells were harvested at Day 3 and Day 6. A heat map and a bar graph of normalized relative expression for GSU cells at Day 3 are shown in FIGS. 6A and 6B, respectively. A heat map and a bar graph of normalized relative expression for GSU cells at Day 6 are shown in FIGS. 7A and 7B, respectively. A heat map and a bar graph of normalized relative expression for A549 cells at Day 3 are shown in FIGS. 8A and 8B, respectively. A heat map and a bar graph of normalized relative expression for A549 cells at Day 6 are shown in FIGS. 9A and 9B, respectively. A heat map and a bar graph of normalized relative expression for Flo1 cells at Day 3 are shown in FIGS. 10A and 10B, respectively. A heat map and a bar graph of normalized relative expression for Flo1 cells at Day 6 are shown in FIGS. 11A and 11B, respectively.

Most of the targets displayed on the heat maps show an increase in expression after exposure to LD-XRT in vitro. This confirms findings from RNA sequencing data in at least the three cell lines discussed in this example.

All of the methods disclosed and claimed herein can be made and executed without undue experimentation in light of the present disclosure. While the compositions and methods of this invention have been described in terms of particular embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method, comprising:
administering low-dose radiation to a patient suffering from a cancer, whereby expression of a target on a surface of a cancer cell of the caner of the patient increases after the low-dose radiotherapy; and
administering a cell-based target-specific cancer therapy to the patient, wherein a cell of the cancer therapy interacts with the target.

2. The method of claim 1, wherein the cancer is characterized by a solid tumor.

3. The method of claim 1, wherein the target is selected from the group consisting of SCARB2, SERINC1, IL6ST, IL6R, XPC, ITGAV, MR1, and DCT.

4. The method of claim 1, wherein the administering comprises delivering from one dose to five doses of the low-dose radiation.

5. The method of claim 4, wherein the administering comprises delivering from two to three doses of the low-dose radiation.

6. The method of claim 1, wherein the cell of the cell-based target-specific cancer therapy is selected from the group consisting of a chimeric antigen receptor (CAR) T cell, a CAR natural killer (NK) cell, a T cell receptor (TCR) engineered T cell, and two or more thereof.

7. The method of claim 1, further comprising:
administering an additional cancer treatment modality other than the cell-based target-specific cancer therapy to the patient.

8. The method of claim 7, wherein the additional cancer treatment modality is selected from the group consisting of surgical resection, chemotherapy, immunotherapy, checkpoint inhibitor therapy, oncolytic virus therapy, thermal therapy, and two or more thereof.

9. A method, comprising:
administering low-dose radiation to a patient suffering from a cancer, whereby expression of a target inside a cancer cell of the cancer of the patient increases after the low-dose radiotherapy; and
administering a small molecule cancer therapy to the patient, wherein the small molecule cancer therapy interacts with the target.

10. The method of claim 9, wherein the cancer is characterized by a solid tumor.

11. The method of claim 9, wherein the target is selected from the group consisting of CDKN1A (p21), DNAJB4, FNIP2, GPRASP2, GSKIP, GUCD1, HIST1H2AC, HIST1H2BC, KLF11, LIG4, MDM2, and NBR1.

12. The method of claim 9, wherein the administering comprises delivering from one dose to five doses of the low-dose radiation.

13. The method of claim 12, wherein the administering comprises delivering from two to three doses of low-dose radiation.

14. The method of claim 9, further comprising:
administering, to the patient, an additional cancer treatment modality other than the small molecule cancer therapy.

15. The method of claim 14, wherein the additional cancer treatment modality is selected from the group consisting of surgical resection, immunotherapy, checkpoint inhibitor therapy, oncolytic virus therapy, thermal therapy, and two or more thereof.

16. A kit, comprising:
a cell-based cancer therapy composition specific for an extracellular target or an intracellular target of a cancer cell of a patient suffering from cancer; and
instructions for the use of the cancer therapy composition in a method comprising administering low-dose radiation to a patient suffering from a cancer, whereby expression of a target on a surface of a cancer cell of the cancer of the patient increases after the low-dose radiation; and administering the cell-based target-specific cancer therapy to the patient, wherein a cell of the cell-based cancer therapy interacts with the target.

17-20. (canceled)

21. The method of claim 1, wherein each dose of the low-dose radiation administered to the patient is about 0.1 Gy to about 10 Gy.

22. The method of claim 4, wherein each dose of the low-dose radiation administered to the patient is about 0.5 Gy to about 2 Gy.

23. The method of claim 9, wherein each dose of the low-dose radiation administered to the patient is about 0.1 Gy to about 10 Gy.

24. The method of claim 12, wherein each dose of the low-dose radiation administered to the patient is about 0.5 Gy to about 2 Gy.
